(19) United States
(12) Patent Application Publication
(10) Pub. No.: US 2025/0319066 A1
(43) Pub. Date: Oct. 16, 2025

(54) INJECTABLE DMT FORMULATIONS

(71) Applicant: Babywatch, Inc., San Francisco, CA (US)

(72) Inventors: Ede FRECSKA, Debrecen (HU); Josipa VLAINIC, Sesvete (HR); Sandro MUR, Samobor (HR)

(21) Appl. No.: 19/177,076

(22) Filed: Apr. 11, 2025

Related U.S. Application Data
(60) Provisional application No. 63/633,259, filed on Apr. 12, 2024.

(51) Int. Cl. A61K 31/4045; A61K 47/02; A61K 47/12; A61K 47/18; A61K 47/32; A61K 47/40; A61K 47/44

(52) U.S. Cl. CPC A61K 31/4045; A61K 47/02; A61K 47/12; A61K 47/18; A61K 47/32; A61K 47/40; A61K 47/44

(57) ABSTRACT

Aspects of the invention can include a composition including an N,N-Dimethyltryptamine base in the range of 0.5-5 mg/mL, a buffer component with acidic acid in the range of 2-15 mM, a cyclodextrin component in the range of 5-15 mM, and a solubilizer component in the range of 1-5 mM; The composition can also include a tonicity agent, wherein the tonicity agent causes an osmolarity of the composition to be in the range of 250-600 mOsm/kg, wherein the composition has a pH in the range of 3-6.

### FIELD OF THE INVENTION

The present invention is directed to a ready-to-use, liquid injectable Dimethyltryptamine formulation, method of formulation preparation, and its uses.

### BACKGROUND

N,N-Dimethyltryptamine (Dimethyltryptamine) belongs to the class of serotonergic binding molecules, a class of molecules that share both structural and functional similarities with serotonin and bind to 5-HT. Dimethyltryptamine is endogenously produced in animals, including humans. Dimethyltryptamine and its derivatives were shown experimentally to bind non-selectively to the 5-HT family of receptors. Additionally, Dimethyltryptamine and its derivatives show binding affinity to other receptors as well including dopamine D1, α1-adrenergic, α2-adrenergic, imidazoline-1, and σ1 receptors as well as serotonin transporter (SERT).

Dimethyltryptamine and its derivatives have been repeatedly shown to play the crucial role and possible regulation of the diverse psychological clinical disorders such as depression, anxiety, substance abuse and others. Moreover, Dimethyltryptamine and its derivatives have shown physiological effects and regulations such as anti-hypoxic, role in neurogenesis, immunomodulatory effect that may contribute to the significant anti-inflammatory and tissue regeneration effects, potential therapeutic effects regarding brain injury and stroke and others.

Therefore, as the potential of Dimethyltryptamine and its derivatives for therapeutic usages increases supported by the number of experiments, there is a need in the art for a ready-to-use N,N-Dimethyltryptamine and its derivatives formulation that is stable for a prolonged period under predefined storage conditions.

### SUMMARY

Aspects of the invention relate to a ready-to-use formulation of N,N-Dimethyltryptamine base, its pharmaceutically acceptable salts, or derivatives thereof, as well as products comprising the formulation.

In some aspects of the invention, a composition can include an N,N-Dimethyltryptamine base in the range of 0.5-5 mg/mL; a buffer component with acidic acid in the range of 2-15 mM; a cyclodextrin component in the range of 5-15 mM; a solubilizer component in the range of 1-5 mM; and a tonicity agent, wherein the tonicity agent causes an osmolarity of the composition to be in the range of 250-600 mOsm/kg, wherein the composition has a pH in the range of 3-6.

In some aspects of the invention, a composition can include a composition including an N,N-Dimethyltryptamine base in an amount of 1 mg/mL; a buffer component with acidic acid in an amount of 5 mM; a cyclodextrin component in an amount of 8 mM; a solubilizer component in an amount of 1%; and a tonicity agent, wherein the tonicity agent causes an osmolarity of the composition to be in the amount of 400 mOsm/kg, wherein the composition has a pH in the amount of about 5.

In some aspects of the invention, a composition can include a composition including an N,N-Dimethyltryptamine base in the amount of 2.5 mg/mL; a glutamic acid buffer component having an acidic acid amount of 5 mM; a povidone component in the amount of 8 mM; a solubilizer component at an amount of 1.5%; and a tonicity agent, wherein the tonicity agent causes an osmolarity of the composition to be about 400 mOsm/kg, wherein the composition has a pH of about 5.

In some aspects of the invention, a composition can include a composition including an N,N-Dimethyltryptamine base in the amount of 5 mg/mL; a glutamic acid buffer component having an acidic acid amount of 5 mM; a cyclodextrin component in the amount of 1 mM; a povidone component in the amount of 8 mM; a solubilizer component at an amount of 2%; and a tonicity agent, wherein the tonicity agent causes an osmolarity of the composition to be about 400 mOsm/kg, wherein the composition has a pH of about 5.

In some aspects of the invention, a composition can include a method including combining components into a composition, the ingredients including: an N,N-Dimethyltryptamine base in the range of 0.5-5 mg/mL; a buffer component with acidic acid in the range of 2-15 mM; a cyclodextrin component in the range of 5-15 mM; a solubilizer component in the range of 1-5 mM; and a tonicity agent, wherein the tonicity agent causes an osmolarity of the composition to be in the range of 250-600 mOsm/kg, wherein the composition has a pH in the range of 3-6.

In some aspects of the invention, a composition can include a method including combining components into a composition, the ingredients including: an N,N-Dimethyltryptamine base in an amount of 1 mg/mL; a buffer component with acidic acid in an amount of 5 mM; a cyclodextrin component in an amount of 8 mM; a solubilizer component in an amount of 1-5 mM; and a tonicity agent, wherein the tonicity agent causes an osmolarity of the composition to be in the range of 250-600 mOsm/kg, wherein the composition has a pH in the range of 3-6.

### DETAILED DESCRIPTION

It should be noted that the invention is not limited examples provided herein, which are referred to for purposes of illustration only.

In this regard, in the descriptions herein, certain specific details are set forth in order to provide a thorough understanding of various embodiments or aspects of the invention. However, one skilled in the art will understand that the invention may be practiced at a more general level without one or more of these details.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, characteristics of different embodiments may be combined in any suitable manner to form one or more other embodiments. In addition, the terms “aspect” or “aspects” can be used interchangeably with the term embodiment or embodiments.

According to aspects of the present invention, degradation of N,N-Dimethyltryptamine is minimized, and the liquid formulation is stable for a prolonged period under refrigerated and controlled room temperature storage conditions.

In one aspect, pH of the formulation is from 3.0 to 6.0. In one aspect, ready-to-use formulation is in a unit dosage form. In one aspect, the concentration of N,N-Dimethyltryptamine in the formulation is from 0.01 to 20.0 mg/ml. In one aspect, the pharmaceutical formulation of the present disclosure is in aqueous form.

The inventors of the present invention have unexpectedly found that when N,N-Dimethyltryptamine is formulated in formulations according to the present disclosure, formation of N,N-Dimethyltryptamine degradation products is minimized, and accordingly, such formulations exhibit prolonged stability and provide more flexible storage conditions and handling when stored under refrigerated and controlled room temperature. In some aspects of the invention, the components and amounts used in the composition can be deemed critical to achieve these benefits of the invention.

The pharmaceutical formulations according to the present disclosure may be used but are not restricted to treat obsessive-compulsive disorder (OCD), anorexia nervosa, bulimia, binge eating disorder (BED), major depressive disorder, persistent depressive disorder, stroke, bipolar disorder, bipolar depression, generalized anxiety disorder, panic disorder, depression in terminally ill patients, social anxiety disorder, post-traumatic stress disorder (PTSD), post-partum depression, substance abuse, an avolition disorder, cancer related anxiety general, alcohol use disorder and treatment resistance depression.

As used herein, the term “derivative” defines either a Dimethyltryptamine base molecule, either pharmaceutically acceptable salts thereof or analogs thereof including any mixture thereof. The term derivative also includes, but is not restricted to, the 5-MeO-Dimethyltryptamine base or any pharmaceutically acceptable salts thereof or analogues thereof including any mixture thereof. The term derivative also includes any isotopically labelled compound of the Dimethyltryptamine and 5-MeO-Dimethyltryptamine bases, pharmaceutically acceptable salts thereof or analogs thereof including any mixture thereof.

As used herein, the term “analog” or “analog compound” defines chemical compounds that have similar physical, chemical, biochemical, or pharmacological properties.

As used herein, the term “pharmaceutically acceptable salt” refers to a salt that is safe for use in pharmaceutical formulations and does not introduce any harmful effects to the patient when administered according to prescribed dosage regimens. Such a salt may be prepared from bases, including inorganic acids, inorganic bases, organic acids, inorganic bases, solvates, hydrates, and clathrates thereof, pamoate, or any mixture thereof.

As used herein, the term “ready-to-use” refers to a formulation comprising active pharmaceutical ingredients dissolved or suspended in an aqueous solution, wherein said formulation is pre-prepared and packaged in a suitable container in a manner that requires no further manipulation or preparation prior to administration. The packaging is designed to maintain the integrity, potency, and sterility of the product throughout its shelf life, protecting it from environmental factors such as light, moisture, and microbial contamination. Such packaging may include vials, ampules, prefilled syringes, or other suitable containers sealed in a manner to prevent tampering and preserve product quality until use.

The present disclosure relates to ready-to-use N,N-Dimethyltryptamine formulations and products comprising the formulations.

According to the present invention, degradation of N,N-Dimethyltryptamine is minimized and the and the liquid formulation is stable for a prolonged period under refrigerated and controlled room temperature storage conditions.

In one aspect, the pH of the formulation is from 3.0 to 6.0.

In one aspect, the pH of the formulation is from 3.0 to 5.0.

In one aspect, the pH of the formulation is from 3.0 to 4.0.

In one aspect, the pH of the formulation is 3.0, 3.1, 3.2, 3.3, 3.4, 3.5, 3.6, 3.7, 3.8, 3.9 and 4.0.

In one aspect, composition is in unit dosage form.

As used herein, “pH” is the conventional measurement unit of hydrogen ion activity in a solution at controlled room temperature unless another temperature is specified.

It was found that when N,N-Dimethyltryptamine is formulated in formulations according to the present disclosure, formation of degradation products is minimized, and accordingly, such formulations exhibit prolonged chemical stability.

The formulations according to the present disclosure can be stored for a certain time at 2-8° C. and also under controlled room temperature conditions.

As used herein, the term “controlled room temperature” has the meaning of a controlled room temperature as set in United States Pharmacopeia standard USP <659>, i.e., from 20 to 25° C.

As used herein, the term “stable” means that the compositions meet one or more of the following criteria:
(i) The pharmaceutical formulation exhibits an acceptable N,N-Dimethyltryptamine assay drop after a certain period; and/or
(ii) the pharmaceutical formulation exhibits an acceptable amount of total impurities being formed after a certain period compared to the amount of total impurities present at the beginning of the period, expressed as a percentage or area-percentage; and/or
(iii) the composition retains a desirable appearance such as clarity, color, and no visible particles (i.e., precipitate free or particle free composition) over a certain period.

As used herein, an acceptable amount of active ingredient degraded after a certain period (i.e., a drop in N,N-Dimethyltryptamine assay) is calculated as a difference in N,N-Dimethyltryptamine assay between assay determined immediately after formulation preparation and assay determined at specific stability testing point e.g., after 1 week, 2 weeks, 1 month, 2 months, 3 months, 6 months etc. The N,N-Dimethyltryptamine assay is analyzed, for example, by liquid chromatography, e.g., HPLC, UPLC.

In one aspect of the invention, a ready-to-use composition according to the present disclosure is stable under controlled room temperature conditions for a certain period.

In one aspect, the compositions are stable for at least 14 days, at least 1 month, at least 2 months, at least 3 months, at least 6 months, at least 12 months when stored under controlled room temperature conditions.

In one aspect, the compositions are stable for at least 14 days, at least 1 month, at least 2 months, at least 3 months, at least 6 months, at least 9 months, or at least 12 months, or at least 15 months when stored under 2-8° C. conditions.

In a specific aspect, as used herein, the term “stable” is defined as no more than 10% of drop of N,N-Dimethyltryptamine assay in the pharmaceutical formulation, analyzed by liquid chromatography.

For example, a stable composition can be one which has not more than 1%, 2%, 3%, 4%, 5%, 6%, 7%, 8%, 9%, 10% drop of N,N-Dimethyltryptamine assay after a predetermined time period analyzed by liquid chromatography.

In a specific aspect of the invention, the term “stable” is defined as not more than 10% of total impurities of N,N-Dimethyltryptamine in the pharmaceutical formulation are formed, analyzed by liquid chromatography. A drop is determined by assaying the amount of N,N-Dimethyltryptamine in a given amount of sample immediately after it is formulated, and then measuring the amount of N,N-Dimethyltryptamine in the same amount of sample at a later point in time.

For example, a stable composition can be one which has not more than 1%, 2%, 3%, 4%, 5%, 6%, 7%, 8%, 9%, 10% of N,N-Dimethyltryptamine total impurities after a predetermined time period analyzed by liquid chromatography.

As used herein, “stable” may also be defined as a formulation having no visible particles (free of particles or particles/precipitate free) in the pharmaceutical formulation after a predefined time.

As used herein, the terms “pharmaceutical composition” or “pharmaceutically acceptable composition” have the meaning of any composition suitable and intended for in vivo use. For example, for use in administration to a patient or a subject. As used herein, the terms “patient” and “subject” are interchangeable and refer to any human or animal individual who is receiving a composition as described herein.

As used herein, the terms “pharmaceutical composition”, “pharmaceutically acceptable composition”, “pharmaceutical formulation”, “composition” and “formulation” are used interchangeably.

In one aspect, the concentration of N,N-Dimethyltryptamine in the formulation is from 0.01 mg/ml to 20 mg/ml.

In one aspect, the concentration of N,N-Dimethyltryptamine in the formulation is from 0.1 mg/ml to 10 mg/ml.

In one aspect, the concentration of N,N-Dimethyltryptamine in the formulation is from 0.5 mg/ml to 5 mg/ml.

In one aspect, N,N-Dimethyltryptamine is in the form of a base.

In one aspect, N,N-Dimethyltryptamine base is dissolved using buffers at the pH range from 3.0-6.0. Buffers include, but are not limited to, aspartic acid, glutamic acid, maleic acid, glycolic acid, glyceric acid, malic acid, methionine, lipoic acid, picolinic acid and appropriate base e.g., sodium hydroxide, ammonium hydroxide, sodium bicarbonate, or similar.

In one aspect, the concentration of the acidic component of the buffer is from 1 mM to 20 mM. In one aspect, the concentration of the acidic component of the buffer is from 2 mM to 15 mM. In one aspect, the concentration of the acidic component of the buffer is from 5 mM to 10 mM.

In one aspect of the invention, the formulation comprises a tonicity agent.

As used herein, the term “tonicity” or effective osmolality describes the capacity of particles in solution to effect water movement across a semipermeable membrane like the cell membrane. In turn, as used herein, the term “tonicity agent” describes substances which are used to adjust the tonicity of the solution.

In one aspect of the invention, tonicity agents an include, but are not limited to, Sodium Chloride, Potassium Chloride, Dextrose, Glycerol, Glycine, Sorbitol and Mannitol.

In one aspect of the invention, a tonicity agent is used in the formulation to adjust the osmolality of the solution in the range from 200 mOsm/kg to 600 mOsm/kg.

In one aspect of the invention, a tonicity agent is used in the formulation to adjust the osmolality of the solution in the range from 250 mOsm/kg to 350 mOsm/kg.

In another aspect of the invention, a N,N-Dimethyltryptamine base is dissolved using solubilizers, and pH is adjusted in any suitable manner. The pH may be adjusted with one or more pH adjusting agents, which may be selected from mineral acids, organic acids, weak and strong bases, and salts and derivatives thereof. Examples of pH adjusting agents can include, but are not limited to, hydrochloric acid, phosphoric acid, sulfuric acid, acetic acid, succinic acid, lactic acid, citric acid, phenolic acid, aspartic acid, glutamic acid, maleic acid, glycolic acid, glyceric acid, malic acid, methionine, lipoic acid, picolinic acid or appropriate base e.g., sodium hydroxide ammonium hydroxide, sodium bicarbonate.

In one aspect, the solubilizer is cyclodextrin.

In one aspect, cyclodextrin is hydroxypropyl beta-cyclodextrin, sulfobutylether beta-cyclodextrin, or a combination thereof.

In one aspect, the concentration of at least one cyclodextrin is from 1 mM to 20 mM.

In one aspect, the solubilizer is poloxamer. As is known in the art, Poloxamers are nonionic triblock copolymers composed of a central hydrophobic chain of polyoxypropylene (poly(propylene oxide)) flanked by two hydrophilic chains of polyoxyethylene (poly(ethylene oxide)). Different poloxamers are identified by numbers as is known in the art.

In one aspect of the invention, the poloxamer can be 188, 338, 407, or a combination thereof.

In one aspect, the concentration of at least one poloxamer is from 1 mM to 20 mM.

In one aspect, the solubilizer is povidone.

In one aspect, povidone is K12, 17, 30, or a combination thereof.

In one aspect, the concentration of at least one povidone is from 1 mM to 20 mM.

In one aspect, the solubilizer is Polyoxyl 40 Hydrogenated castor oil.

In one aspect, the concentration of Polyoxyl 40 Hydrogenated castor oil is from 0.5% to 5%.

In one aspect, formulation comprises tonicity agent.

In one aspect, tonicity agents include, but not limited to, Sodium Chloride, Potassium Chloride, Dextrose, Glycerol, Glycine, Sorbitol and Mannitol.

In one aspect, a tonicity agent is used in the formulation to adjust the osmolality of the solution in the range from 200 Osm/kg to 600 Osm/kg.

In one aspect, a tonicity agent is used in the formulation to adjust the osmolality of the solution in the range from 250 Osm/kg to 350 Osm/kg.

In one aspect, formulation may include of 0.5-5 mg/mL N,N-Dimethyltryptamine base, buffer with acidic acid component from 2-15 mM, cyclodextrin from 5-15 mM, solubilizer component from 1-5 mM, tonicity agent to result in osmolarity 250-600 mOsm/kg, at the pH from 3-6.

In one aspect, formulation may include 1 mg/mL N,N-Dimethyltryptamine base, preferably maleic acid buffer with acidic acid component at 5 mM, cyclodextrin at 8 mM (preferably sulfobutylether beta-cyclodextrin), solubilizer component Polyoxyl 40 Hydrogenated castor oil at 1% and tonicity agent sodium chloride to result in osmolarity around 400 mOsm/kg at the pH around 5.

In one aspect, formulation may include of 2.5 mg/mL N,N-Dimethyltryptamine base, preferably glutamic acid buffer with acidic acid component at 5 mM, povidone at 8 mM (preferably povidone K12), solubilizer component Polyoxyl 40 Hydrogenated castor oil at 1.5% and tonicity agent dextrose to result in osmolarity around 400 mOsm/kg at the pH around 5.

In one aspect, formulation may include 5 mg/mL N,N-Dimethyltryptamine base, preferably glutamic acid buffer with acidic acid component at 5 mM, cyclodextrin at 1 mM (preferably sulfobutylether beta-cyclodextrin), povidone at 8 mM (preferably povidone K12), solubilizer component Polyoxyl 40 Hydrogenated castor oil at 2% and tonicity agent dextrose to result in osmolarity around 400 mOsm/kg at the pH around 5.

As used herein, the terms “around” and “about” is interpreted to mean within a range that extends up to ±10% of the stated value. The terms “around” and “about” are used interchangeably herein.

In one aspect, N,N-Dimethyltryptamine is in the form of a salt. A N,N-Dimethyltryptamine salt can be salts of alkyl or aryl carboxylic acids, mineral acids.

In one aspect, formulation comprises a buffer at the pH range from 3.0-6.0. Buffers include, but are not limited to, aspartic acid, glutamic acid, maleic acid, glycolic acid, glyceric acid, malic acid, methionine, lipoic acid, picolinic acid and appropriate base e.g., sodium hydroxide, ammonium hydroxide, sodium bicarbonate, or similar.

In one aspect, the pH of the formulation is from 3.0 to 6.0.

In one aspect, the pH of the formulation is from 3.0 to 5.0.

In one aspect, the pH of the formulation is from 3.0 to 4.0.

In one aspect, the pH of the formulation is 3.0, 3.1, 3.2, 3.3, 3.4, 3.5, 3.6, 3.7, 3.8, 3.9 and 4.0.

In one aspect, the concentration of the acidic component of the buffer is from 1 mM to 20 mM.

In one aspect, the concentration of the acidic component of the buffer is from 2 mM to 15 mM.

In one aspect, the concentration of the acidic component of the buffer is from 5 mM to 10 mM.

In one aspect, formulation comprises tonicity agent. As used herein the term “tonicity” or effective osmolality describes the capacity of particles in solution to effect water movement across a semipermeable membrane like the cell membrane. As used herein the term “tonicity agent” describes substances which are used to adjust the tonicity of the solution.

In one aspect, tonicity agents include, but not limited to, Sodium Chloride, Potassium Chloride, Dextrose, Glycerol, Glycine, Sorbitol and Mannitol.

In one aspect, a tonicity agent is used in the formulation to adjust the osmolality of the solution in the range from 250 mOsm/kg to 600 mOsm/kg.

In one aspect, a tonicity agent is used in the formulation to adjust the osmolality of the solution in the range from 250 mOsm/kg to 350 mOsm/kg.

In one aspect, formulation may include of 0.5-5 mg/mL N,N-Dimethyltryptamine salt, buffer with acidic acid component from 2-15 mM, tonicity agent to result in osmolarity 250-600 mOsm/kg, at the pH from 3-6.

In one aspect, formulation may include 1 mg/mL N,N-Dimethyltryptamine dissolved in the form of salt (preferably fumarate), aspartic acid buffer with acidic acid component at 5 mM and tonicity agent sodium chloride to result in osmolarity around 400 mOsm/kg at the pH around 3.2.

In one aspect, formulation may include 2.5 mg/mL N,N-Dimethyltryptamine dissolved in the form of salt (preferably fumarate), aspartic acid buffer with acidic acid component at 5 mM and tonicity agent sodium chloride to result in osmolarity around 400 mOsm/kg at the pH around 3.2.

In one aspect, formulation may include 5 g/mL N,N-Dimethyltryptamine dissolved in the form of salt (preferably fumarate), aspartic acid buffer with acidic acid component at 5 mM and tonicity agent sodium chloride to result in osmolarity around 400 mOsm/kg at the pH around 3.2.

In one aspect, the pharmaceutical formulation is aqueous.

As used herein, the term “aqueous composition”, “aqueous solution” or “aqueous” is understood as any composition in which the solvent is water (e.g., water for injection). Accordingly, aqueous compositions include compositions comprising water in concentration of at least 80% v/v, at least 85% v/v, at least 90% v/v, at least 95% v/v or at least 99% v/v.

In one aspect, aqueous composition of N,N-Dimethyltryptamine comprises 90% or more water.

In an aspect, pharmaceutical formulation may comprise water or standard diluents for parenteral use, such as Water for Injection, 0.9% Sodium Chloride for injection, 5% Dextrose, or other suitable diluents.

This disclosure also provides a process for manufacturing any disclosed pharmaceutical formulation. In various embodiments, the process may comprise the steps of dissolving predefined excipients and N,N-Dimethyltryptamine in water or predefined diluents and filling the predefined container with produced formulation. In some cases, pH can be adjusted to achieve the desired pH range.

In another aspect, the pharmaceutical formulation may be manufactured by any process known to the person skilled in the art.

In one aspect, the liquid formulation of N,N-Dimethyltryptamine is packaged into a suitable container. The container may be a syringe, a vial, an ampule, a blow-fill-seal container or an IV bag. In one aspect, the container is a single unit dose container. In one aspect, the container is a single unit dosage container for IV administration.

In one aspect, the volume of the container is from 1 ml to 100 ml.

In one aspect, the volume of the container is from 20 ml to 100 ml.

In one aspect, the container may, optionally, further comprise an overwrap. In one aspect, the overwrap may comprise gas-barrier and/or light barrier material.

In one aspect, the overwrap is made of aluminum.

The formulation disclosed herein may be sterilized by known means. Such known means in the art comprise sterile filtration, heat treatment and/or irradiation.

In one aspect, the present disclosure provides a method of treating humans by administering an effective dose of N,N-Dimethyltryptamine by an IV route.

By way of aspects of the present invention, degradation of N,N-Dimethyltryptamine is beneficially minimized and the liquid formulation is stable for a prolonged period under refrigerated and controlled room temperature storage conditions.

It should be obvious to one of ordinary skill in the art that subsets or combinations of various embodiments described above provide further embodiments.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

What is claimed is:

1. A composition comprising:
an N,N-Dimethyltryptamine base in the range of 0.5-5 mg/mL;
a buffer component with acidic acid in the range of 2-15 mM;
a cyclodextrin component in the range of 5-15 mM;
a solubilizer component in the range of 1-5%; and
a tonicity agent, wherein the tonicity agent causes an osmolarity of the composition to be in the range of 250-600 mOsm/kg,
wherein the composition has a pH in the range of 3-6.

2. A composition comprising:
an N,N-Dimethyltryptamine base in an amount of 1 mg/mL;
a buffer component with acidic acid in an amount of 5 mM;
a cyclodextrin component in an amount of 8 mM;
a solubilizer component in an amount of 1%; and
a tonicity agent, wherein the tonicity agent causes an osmolarity of the composition to be in the amount of 400 mOsm/kg,
wherein the composition has a pH in the amount of about 5.

3. The composition of claim 2, wherein
the buffer component includes maleic acid.

4. The composition of claim 2, wherein
the solubilizer component is Polyoxyl 40 Hydrogenated castor oil.

5. The composition of claim 2, wherein
the tonicity agent is sodium chloride.

6. A composition comprising:
an N,N-Dimethyltryptamine base in the amount of 2.5 mg/mL;
a glutamic acid buffer component having an acidic acid amount of 5 mM;
a povidone component in the amount of 8 mM;
a solubilizer component at an amount of 1.5%; and
a tonicity agent, wherein the tonicity agent causes an osmolarity of the composition to be about 400 mOsm/kg,
wherein the composition has a pH of about 5.

7. The composition of claim 6, wherein
the povidone component is povidone K12.

8. The composition of claim 6, wherein
the solubilizer component is Polyoxyl 40 Hydrogenated castor oil.

9. A composition comprising:
an N,N-Dimethyltryptamine base in the amount of 5 mg/mL;
a glutamic acid buffer component having an acidic acid amount of 5 mM;
a cyclodextrin component in the amount of 1 mM;
a povidone component in the amount of 8 mM;
a solubilizer component at an amount of 2%; and
a tonicity agent, wherein the tonicity agent causes an osmolarity of the composition to be about 400 mOsm/kg,
wherein the composition has a pH of about 5.

10. The composition of claim 9, wherein
the cyclodextrin component is sulfobutylether beta-cyclodextrin.

11. The composition of claim 6, wherein
the povidone component is povidone K12.

12. The composition of claim 6, wherein
the solubilizer component is Polyoxyl 40 Hydrogenated castor oil.

13. A method of forming a formulation, the method including:
combining components into a composition, the ingredients including:
an N,N-Dimethyltryptamine base in the range of 0.5-5 mg/mL;
a buffer component with acidic acid in the range of 2-15 mM;
a cyclodextrin component in the range of 5-15 mM;
a solubilizer component in the range of 1%; and
a tonicity agent, wherein the tonicity agent causes an osmolarity of the composition to be in the range of 250-600 mOsm/kg,
wherein the composition has a pH in the range of 3-6.

14. A method of forming a formulation, the method including:
combining components into a composition, the ingredients including:
an N,N-Dimethyltryptamine base in an amount of 1 mg/mL;
a buffer component with acidic acid in an amount of 5 mM;
a cyclodextrin component in an amount of 8 mM;
a solubilizer component in an amount of 1%; and
a tonicity agent, wherein the tonicity agent causes an osmolarity of the composition to be in the range of 400 mOsm/kg,
wherein the composition has a pH in the amount of about 5.

15. The method of claim 14, wherein
the buffer component includes maleic acid.

16. The method of claim 14, wherein
the solubilizer component is Polyoxyl 40 Hydrogenated castor oil.

17. The method of claim 14, wherein
the tonicity agent is sodium chloride.

18. The method of claim 14, wherein
the composition is in an aqueous form.
